(19) United States
(12) Patent Application Publication
(10) Pub. No.: US 2022/0008525 A1
(43) Pub. Date: Jan. 13, 2022

(54) CANCER SPECIFIC FRAMESHIFT VACCINES

(71) Applicant: Arizona Board of Regents on Behalf of Arizona State University, Scottsdale, AZ (US)

(72) Inventor: Stephen Albert JOHNSTON, Tempe, AZ (US)

(21) Appl. No.: 17/310,461

(22) PCT Filed: Feb. 7, 2020
(86) PCT No.: PCT/US20/17340

§ 371 Date: Aug. 4, 2021

Related U.S. Application Data
(60) Provisional application No. 62/802,672, filed on Feb. 7, 2019.

(51) Int. Cl. A61K 39/00; A61P 35/04; A61K 45/06; A61K 39/39

(52) U.S. Cl. CPC A61K 39/0011; A61P 35/04; A61K 45/06; A61K 39/39; A61K 2039/852; A61K 2039/86; A61K 2039/80; A61K 2039/804; A61K 2039/812

(57) ABSTRACT

A method of producing a vaccine for a cancer and/or tumor and stage of interest is disclosed. The method includes identifying a first population of peptides that are immunoreactive with a set of biological samples obtained from a set of test subjects that have been identified as having the cancer and/or tumor of interest, and preparing a cancer vaccine composition for the cancer of interest, wherein the cancer vaccine composition comprises a second population of peptides comprising one or more peptides in the first population or a nucleic acid sequence encoding the one or more peptides, thereby producing the vaccine for the cancer and/or tumor of interest. Also disclosed are vaccine compositions and methods of use thereof.

### CROSS REFERENCE TO RELATED APPLICATION

This disclosure claims the benefit of the earlier filing date of U.S. Provisional Application No. 62/802,672 filed on Feb. 7, 2019, which is hereby incorporated by reference in its entirety.

### FIELD OF DISCLOSURE

This disclosure concerns cancer and, more specifically, the development of vaccines for cancer.

### BACKGROUND

Cancer is the second leading cause of death in the US. For early stage cancers (those that have not spread to the lymph nodes and are non-metastatic) surgical removal is a very effective treatment. However, for more advanced cases, standard, non-specific cancer treatments (chemo and radiotherapy) are used. These treatments affect many healthy cells and result in elevated toxicity. The idea of employing the adaptive immune system to kill cancer cells without harming normal cells has been a goal for many decades. However, tumor antigen identification and its translation to immunotherapy still face many problems. Personal vaccines based on sequencing each patient's tumor to discover neoantigens to improve therapy have been investigated, but are too expensive and complicated to produce to be adopted. Therefore, vaccines which are therapeutically effective and can be produced efficiently are desired.

### SUMMARY

Disclosed herein are methods of producing a vaccine for a cancer and/or tumor of interest, comprising: identifying a first population of peptides that are immunoreactive with a set of biological samples obtained from a set of test subjects that have been identified as having the cancer and/or tumor and stage of interest; and preparing a cancer vaccine composition for the cancer of interest, wherein the cancer vaccine composition comprises a second population of peptides comprising one or more peptides in the first population or a nucleic acid sequence encoding the one or more peptides, thereby producing the vaccine for the cancer and/or tumor of interest.

In some embodiments, the second population of peptides is a subpopulation of the first population.

In some embodiments, identifying first population of peptides comprises determining immunoreactivity of the first population of peptides to the set of biological samples obtained from the set test subjects using antibody reactivity.

In some embodiments, antibody reactivity is detected using an antibody assay comprises ELISA, radio-immunoassay, western blot, surface plasmon resonance, immunostaining, immunoprecipitation, mass spectrometry, phage display, ELISPOT, flow cytometry, cytometric bead array, immunohistochemistry, high density array, microarray, delayed-type hypersensitivity (DTH), and combinations thereof.

In some embodiments, identifying comprises determining immunoreactivity of the first population of peptides to the set of biological samples obtained from the set test subjects using a T cell response.

In some embodiments, T cell response is detected using a proliferation assay, 3H-thymidine assay, BrdU assay, CFSE assay, cytokine secretion assay, ELISA assay, ELISPOT assay, intracellular staining assay, quantitative rtPCR assay, cytometric bead array assay, cytotoxicity assay, 51-chromium assay, degranulation assay, granulysin assay, granzyme A assay, granzyme B assay, and/or perforin assay.

In some embodiments, the first population of peptides comprises peptides encoded by a frameshifted mRNA expressed by a cancer cell.

In some embodiments, the frameshifted mRNA is created in a splicing error or a transcription insertion or deletion error.

In some embodiments, each of the first population of peptides binds to at least one MHC subtype.

In some embodiments, each of the first population of peptides comprises at least one T cell epitope.

In some embodiments, each of the first population of peptides comprises at least one B cell epitope and elicits an antibody response

In some embodiments, the first population of peptides is bound to a substrate.

In some embodiments, the first population of peptides is part of an array or a phage display library.

In some embodiments, the vaccine composition further comprises a pharmaceutically acceptable adjuvant or excipient.

In some embodiments, the vaccine composition further comprises an immune checkpoint inhibitor.

In some embodiments, the set of biological samples obtained from the set test subjects comprises one or more of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor.

In some embodiments, the method further comprises administering an effective amount of the vaccine composition to prevent a cancer or treat a subject in need thereof to treat the cancer and/or tumor.

In some embodiments, treating the cancer and/or tumor comprises reducing tumor size, inhibiting tumor growth, reducing tumor burden, preventing metastasis, increasing survival, or increasing cancer-free survival.

In some embodiments, administering the vaccine composition elicits an immune response in the subject against the cancer.

In some embodiments, the cancer is selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

In some embodiments, a vaccine composition produced by the disclosed methods is provided.

In some embodiments, disclosed are methods of eliciting an immune response in a treatment subject in need thereof, comprising: selecting a treatment subject with cancer or at risk for developing cancer; and administering a disclosed vaccine composition to the subject, thereby eliciting an immune response in the subject in need thereof.

In some embodiments, disclosed are methods of reducing risk of developing cancer in a subject comprising: selecting a treatment subject with cancer or at risk for developing cancer; and administering a disclosed vaccine composition to the subject, thereby reducing risk of developing cancer in the subject.

In some embodiments, disclosed are methods of treating cancer and/or a tumor in a subject comprising: selecting a treatment subject with cancer and/or a tumor and administering a disclosed vaccine composition to the subject, thereby treating the cancer and/or tumor in the subject.

The foregoing and other features of the disclosure will become more apparent from the following detailed description, which proceeds with reference to the accompanying figures.

### BRIEF DESCRIPTION OF THE DRAWINGS

FIG. 1 illustrates specific peptides identified to be associated with 5 different human cancers in accordance with methods for developing cancer frameshift vaccines disclosed herein. Note that GBM has very few DNA neoantigens, but has abundant RNA initiated frameshift neoantigens.

FIG. 2 illustrates the ability to develop a frameshift vaccine for myeloma (MEL) in dogs in accordance with methods for developing vaccines disclosed herein.

FIG. 3 illustrates the ability to develop a frameshift vaccine for breast cancer (BC) in dogs in accordance with methods for developing vaccines as disclosed herein.

FIG. 4 is a schematic illustrating vaccine design in accordance with exemplary embodiments disclosed herein.

FIGS. 5A and 5B illustrate the effectiveness of frameshift, cancer specific vaccines disclosed herein wherein such vaccines confer protection by reducing tumor growth and/or preventing growth at all.

FIGS. 6A and 6B illustrate disclosed frameshift vaccines reduce metastasis.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples:

Adjuvant: A vehicle used to enhance antigenicity; such as a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules.

Administration: The introduction of a composition into a subject by a chosen route. For example, if the chosen route is oral delivery, the composition is administered by introducing the composition into the mouth of the subject.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev Immunol 2:239, 1984). Humanized antibodies and fully human antibodies are also known in the art.

Animal: Living multi-cellular vertebrate organisms, a category that includes, for example, mammals and birds. The term mammal includes both human and non-human mammals. Similarly, the term “subject” includes both human and veterinary subjects.

Array: An arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the subject features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Diagnostic: Identifying the presence or nature of a pathologic condition. Diagnostic methods differ in their sensitivity and specificity. The “sensitivity” of a diagnostic assay is the percentage of diseased subjects who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Epitope: An antigenic determinant. These are particular chemical groups or peptide sequences on a molecule that are antigenic, such that they elicit a specific immune response. An antibody binds a particular antigenic epitope.

Frameshift and Frameshift Peptide: A frameshift in the RNA results from an insertion/deletion or fusion/deletion at the DNA or RNA level that results in the shifting of the reading frame of the ribosome. The resultant peptide is out of frame and does not correspond to a normal life sequence peptide. A Frameshift may also result from the inappropriate initiation of translation by the ribosome. The results of these mistakes are Frameshift Peptides (FSPs).

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or induce/stimulate a B cell response (e.g., antibody production) against the antigen from which the immunogenic peptide is derived.

Label: A detectable compound or composition that is conjugated directly or indirectly to another molecule to facilitate detection of that molecule. Specific, non-limiting examples of labels include fluorescent tags, enzymatic linkages, and radioactive isotopes.

Major Histocompatibility Complex (MHC): A set of genes that code for cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T-cells. MHC molecules mediate interactions of leukocytes, also called white blood cells (WBCs), which are immune cells, with other leukocytes or with body cells. The MHC determines compatibility of donors for organ transplant, as well as one's susceptibility to an autoimmune disease via crossreacting immunization. The human MHC is also called the HLA (human leukocyte antigen) complex (often just the HLA). The MHC in mice is called the Histocompatibility system 2 or just the H-2.

In a cell, protein molecules of the host's own phenotype or of other biologic entities are continually synthesized and degraded. Each MHC molecule on the cell surface displays a molecular fraction of a protein, called an epitope. The presented antigen can be either self or non-self, thus preventing an organism's immune system targeting its own cells. In its entirety, the MHC population is like a meter indicating the balance of proteins within the cell.

The MHC gene family is divided into three subgroups: MHC class I, MHC class II, and MHC class III. Class I MHC molecules have (32 microglobulin subunit which can only be recognised by CD8 co-receptors. Class II MHC molecules have (31 and (32 subunits and can be recognised by CD4 co-receptors. In this way, MHC molecules chaperone which type of lymphocytes may bind to the given antigen with high affinity, since different lymphocytes express different T-Cell Receptor (TCR) co-receptors.

Neoantigen: Peptide sequences that are produced by errors in DNA replication or RNA processing to stimulate an antigenic immune response. Neoantigens are recognized as foreign by the host immune system.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Peptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Set: Two or more things that are thought of as a group and/or that share a common feature. For example, a set of biological samples can be two or more biological samples obtained from subjects having or at risk of having of a particular condition or disease, such as having a particular cancer and/or tumor of interest.

T Cell: A white blood cell critical to the immune response. T cells include, but are not limited to, CD4⁺ T cells and CD8⁺ T cells.

Therapeutically effective amount: A quantity of a specified pharmaceutical or therapeutic agent sufficient to achieve a desired effect in a subject, or in a cell, being treated with the agent. The effective amount of the agent will be dependent on several factors, including, but not limited to the subject or cells being treated, and the manner of administration of the therapeutic composition.

Treatment: A method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for reducing the effects of a cancer is considered to be a treatment if there is a 10% reduction in one or more symptoms of the disease (e.g., tumor size) in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is also understood and contemplated herein that treatment can refer to any reduction in the progression of a disease or cancer. Thus, for example, methods of reducing the effects of a cancer is considered to be a treatment if there is a 10% reduction in the tumor growth rate relative to a control subject or tumor growth rates in the same subject prior to the treatment. It is understood that the reduction can be a 10, 20, 30, 40, 50, 60, 70, 80, 90, 100%, or any amount of reduction in between as compared to native or control levels.

Under conditions sufficient to: A phrase that is used to describe any environment that permits the desired activity. In one example, includes conditions sufficient to induce uptake of a molecule, such as the binding or internalization of an antigen by a cell. In some examples, under conditions sufficient to includes suitable conditions for binding of peptides-antibody on the array and/or any of the in vitro assays, such as those that will be conducted prior to vaccination.

Vaccine: A composition that elicits a prophylactic or therapeutic immune response in a subject. In some cases, the immune response is a protective immune response. Typically, a vaccine elicits an antigen-specific immune response to an antigen of a pathogen, for example, a bacterial or viral pathogen, or to a cellular constituent correlated with a pathological condition, such as cancer. A vaccine may include a polynucleotide, a peptide or polypeptide, a virus, a bacterium, a cell or one or more cellular constituents. In some cases, the virus, bacteria or cell may be inactivated or attenuated to prevent or reduce the likelihood of infection, while maintaining the immunogenicity of the vaccine constituent. The immunogenic material may include live-attenuated or killed microorganisms (such as bacteria or viruses), or antigenic proteins, peptides or DNA derived from them. In some cases, the vaccine is a subunit vaccine, which is an immunizing agent that has been treated to remove traces of nucleic acid (such as viral nucleic acid) so that only protein subunits remain. The subunits have less risk of causing adverse reactions. The vaccine can also be a live vaccine, which is a vaccine prepared from living attenuated organisms or from viruses that have been attenuated but can still replicate in the cells of the host organism.

The immunogenic material for a cancer vaccine may include, for example, a protein or peptide expressed by a tumor or cancer cell. Vaccines may elicit both prophylactic (preventative) and therapeutic responses.

Vector: A vaccine vector is a virus, bacterium, or other microbe, or a nucleic acid, used to deliver an antigen or a gene for an antigen, as part of a vaccine. A nucleic acid vector is a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

Virus: A microscopic infectious organism that reproduces inside living cells. A virus consists essentially of a core of nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so.

“Retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### DESCRIPTION OF SEVERAL EMBODIMENTS

### Introduction

The advent of the use of immunotherapeutics has resulted in emphasis on the importance of neo-epitopes in the immune response to cancer. Neo-epitopes are peptides that are normally not produced to an immunologically recognized level in healthy cells, but are produced in tumors. It had previously been believed that millions of neo-antigens are produced by different tumors, but that very few of them repeated in different tumors, even among tumors the same type. Therefore, it was believed that to develop a cancer vaccine, each tumor would need to be sequenced as well as the non-tumor cells (germline) for each person. These DNA based neoantigens are the basis of personal vaccines.

Because most neo-antigens are the product of point mutations, and only a small portion of these are immunogenic (approximately 3%) in patients, current methods involve application of a complicated informatics approach to predict the best vaccine components. The current basic concept as described in the art is that personal neoantigens in one's tumor are developed by sequencing the patient's tumor. The germline sequence of the patient would also be analyzed for comparison purposes. The developed neoantigens would then be informatically screened to predict which are likely to be immunogenic, and a vaccine manufactured for these particular antigens and delivered in a timely fashion to the patient. In some instances, it is envisioned that this personal vaccine would be delivered with a checkpoint inhibitor. However, personal cancer vaccine development, as well as the checkpoint inhibitor itself, are both expensive. Thus, rather than an expensive personalized vaccine, the inventors have developed method of producing multiple cancer vaccines that are each specific for a particular cancer and/or tumor type, but are generic to the general population. These vaccines could be 1000× less expensive than personal vaccines and therefore available to worldwide application.

To develop cancer vaccines for a specific cancer regardless of the particular subject, the inventors have determined that a smaller subset of RNA based neo-antigens (“frameshift neo-antigens”) can be utilized to develop a cancer vaccine for an individual cancer and/or tumor type. Because the number of RNA based frameshift (FS) neo-antigens is much smaller than the total of all possible DNA based neo-antigens it is possible to produce arrays of these FS neo-antigens and screen these FS array against a set of biological samples obtained from subjects having a particular cancer and/or tumor of interest. The inventors have discovered that tumors make indel variants in most microsatellites in transcription and that mis-splicing is also recurrent at the same genes in tumors. The FS peptides are produced by insertion and deletion mutations (indels) occurring in microsatellite regions or by mis-splicing of RNA. There are approximately 10,000 potential FS peptides transcribed from microsatellites in exons and approximately 220,000 potential FS from mis-splicing. The number of FS to search can be limited by restricting them to ones that are at least 8 amino acids long and/or in oncogenes, essential genes and or highly expressed genes. These genes would be more difficult for a tumor to evolve away from. Therefore, it is only necessary to screen a limited number of approximately 100 to a 220,000 FS peptides to determine the immunogenic components a vaccine for a specific type of cancer and/or tumor.

### Methods of Producing Cancer Vaccines

Disclosed herein is a non-sequencing based method of producing a vaccine for a cancer and/or tumor of interest. The disclosed method includes identifying a first population of peptides that are immunoreactive with a set of biological samples obtained from a set of test subjects that have been identified as having the cancer and/or tumor and stage of interest. In some embodiments, immunoreactivity is determined separately and then the results combined to determine a consensus set of peptides for each cancer and/or tumor type tested. In some embodiments, the samples are pooled and tested concurrently for each cancer and/or tumor type.

In the disclosed method, a set of biological samples is obtained from multiple individuals to define a population of neo-antigens for each cancer and/or tumor type, for example, biological samples are obtained from at least 2, at least 3, at least 4, at least 5, at least 6, at least 7, at least 8, at least 9, at least 10, at least 15, at least 20, at least 25, at least 50, at least 100 or even more individuals identified as having the cancer and/or tumor type of interest. In some examples, controls, such as samples from subjects who do not have cancer are used to determine which FS peptides are specific to the specific cancer and/or tumor type. Once the neo-antigens for cancer and/or tumor type have been identified, the sequences of these peptide are used to prepare a cancer vaccine composition for the cancer and/or tumor type of interest.

In embodiments, the cancer vaccine composition produced includes a second population of peptides comprising one or more peptides in the first population or a nucleic acid sequence encoding the one or more of the peptides in the first population. An important aspect of the disclosed method is that the immunological screens are based on frameshifted peptides that are produced by tumors and are reactive with the immune system, which is not the case with current protocols which generally only sequence the RNA to determine if the gene is transcribed. Further, because the disclosed methods use peptides that are identified from multiple test subjects, the inherent bias of a single individual, such as in a personalized vaccine are diminished. Thus, as opposed to other methods which are tailored to a single subject, the disclosed methods can be used with any individual. Thus disclosed herein are streamlined, cost effective, and efficacious methods to design and produce cancer vaccines for a subject, such as a human or animal subject, such as a vaccine specific for a specific cancer and/or tumor type. Recently there has been interest in developing off the shelf vaccines based on DNA sourced neoantigens, but these only are applicable to a small subset of tumors.

In some embodiments, vaccines designed using methods herein comprise a set of peptides in a number of forms, including as a DNA vaccine, a peptide vaccine, an RNA vaccine, a viral vaccine, a bacterial vaccine or combinations thereof. In some embodiments, vaccines developed herein are loaded or incorporated into antigen presenting cells, such as dendritic cells or macrophages and the loaded cells administered to the subject. In some embodiments, genes encoding the vaccine antigens are used to transform antigen presenting cells through techniques known by those of skill in the art such as CRISPR, transfection, viral or vector transduction, or other gene transfer or incorporation technology. In some embodiments, the disclosed vaccines are administered to a subject to prevent reoccurrence of a cancer and/or tumor.

In some embodiments, the biological sample comprises or is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample is blood, serum, plasma, or saliva. In some embodiments, the biological sample comprises cells selected from B cells, T cells, CD4⁺ T cells, CD8⁺ T cells, Th17 cells, and combinations thereof. In some embodiments, the biological sample comprises an antibody. In some embodiments, all the biological samples tested are the same type. In other embodiments, the biological samples are different types, such as different types of samples listed above.

In some embodiments, the cancer and/or tumor type comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor. Thus, using the methods disclosed herein cancer vaccines can be produced for any of the listed cancer and/or tumor types as well as any that are not listed.

In some embodiments, disclosed herein are array platforms that allow for development of peptides suitable for producing a vaccine to a specific cancer and/or tumor type. The array platforms comprise a plurality of subject features on the surface of the array, for example in addressable locations. Each feature typically comprises a plurality of subject peptides synthesized in situ or spotted on the surface of the array, wherein the molecules are identical within a feature, but the sequence or identity of the molecules differ between features. Such array molecules include the synthesis of large synthetic peptide arrays.

The peptide arrays can include control sequences that match epitopes of well characterized monoclonal antibodies (mAbs). Binding patterns to control sequences and to library peptides can be measured to qualify the arrays and the assay process. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 40 million features on an 8-inch wafer. Photolithographic array synthesis is performed using semiconductor wafer production equipment in a cleanroom to achieve high reproducibility. When the wafer is diced into standard microscope slide dimensions, each slide contains more than 3 million distinct chemical entities.

In some embodiments, arrays with peptide libraries produced by photolithographic technologies disclosed herein are used for immune-based assays. Using a subject's, or multiple subjects, antibody repertoire from a biological sample bound to the arrays, a fluorescence binding profile image of the bound array provides sufficient information to classify which peptides are reactive with an antibody from the subject or from subjects, such as from subjects with a particular disease or condition.

Platforms disclosed herein comprise a selection of frameshift peptides disclosed herein, such as peptides resulting from an insertion or deletion error in transcription of an mRNA or peptides resulting from a splicing error such as a trans-splicing error or a cis-splicing error. In some embodiments, platforms herein comprise frameshift peptides having a sequence selected from all microsatellite frameshifts (MS FS) or MS FS from oncogenes, essential genes, or highly expressed genes.

In some embodiments, the array is a wafer-based, photolithographic, in situ peptide array produced using reusable masks and automation to obtain arrays of scalable numbers of combinatorial sequence peptides. In some embodiments, the peptide array comprises about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more peptides having different sequences. Multiple copies of each of the different sequence peptides can be situated on the wafer at addressable locations known as features.

In some embodiments, the array is a glass slide or nitrocellulose membrane having in vitro synthesized peptides spotted in a predetermined pattern and screened for binding of antibodies in a biological sample, such as obtained from one or more subjects.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

Platforms herein are also contemplated to include peptides in microtiter plates for determining T cell activity in response to frameshift peptides herein. In some embodiments, microtiter plates include but are not limited to 96 well, 384 well, 1536 well, 3456 well, and 9600 well plates. In some embodiments, more than one peptide is present in each well of a microtiter plate, i.e., the peptides are pooled and subject peptides eliciting T cell activity are determined by deconvolution of the positive and negative wells in the T cell assay.

Optionally, it is useful to determine immunogenicity of a candidate frameshift peptide for use in producing a cancer vaccine for a specific cancer and/or tumor type. Immunogenicity, as used herein, refers to the ability of a substance, such as a peptide, to elicit an immune response, such as an antibody response or a T cell response, when administered to a subject, for example, in a vaccine formulation. For subjects with cancer it is the immune response to the tumor. In some embodiments, a peptide that reacts with an antibody or elicits T cell activity in a biological sample from a subject is not immunogenic when administered in a vaccine formulation. In some embodiments, a peptide that reacts with an antibody or elicits T cell activity in a biological sample from a subject is immunogenic when administered in a vaccine formulation Immunogenicity is determined by methods of those of skill in the art including in animal model testing and using in silico prediction of immunogenicity. In silico immunogenicity prediction tools are available for free to the public, for example at the Immune Epitope Database and Analysis Resource (www.iedb.org).

Alternatively, mice, such as mice transgenic for mammalian HLA genes are used to determine the immunogenicity of a candidate frameshift peptide. The candidate frameshift peptide is administered to the transgenic mouse in a vaccine formulation. Response to the vaccine is determined using antibody assays and/or T cell assays described elsewhere herein. In the case of mice that are injected with a tumor or are transgenic to develop tumors, the protection of the frameshift peptide as a vaccine can be determined. Methods herein may include methods of determining the optimal components of a vaccine to be given to a subject to treat a specific cancer in a subject such as a treatment subject. Such methods may include determining whether a candidate vaccine peptide elicits maan immune response in the subject.

The variant peptides comprising the collection for screening, for example, the first population, could be from several sources. They could be peptides known to result from point mutations, frameshifts, deletions/insertions or translocation in tumor DNA. Because these types of mutations are personal and occur infrequently, it would take a large number of peptides to represent all of them. Conventional practice is to determine neo-antigens encoded at the DNA level and then confirm expression at the RNA level. However, the inventors have unexpectedly discovered that variants occur much more frequently at the RNA level only. Since microsatellites in coding regions are predicted and limited in number, one can predict a small set of FS peptides resulting from insertion or deletions during transcription that will produce FS neo-antigens. Therefore, methods herein, in some embodiments, comprise screening frameshift variants formed from 1) insertions or deletions in microsatellites in coding regions or 2) from mis-splicing events either in or between genes that create an out-of-frame fusion. These variants have several attractive features as sources for a cancer vaccine component. First, frameshift variants generally have variant peptide sequences of over more than 8 amino acids. In contrast with point mutations that often only alters one amino acid, a FS variant is completely foreign sequence and therefore is much more likely to be immunogenic. Work indicates that there are only a few thousand frameshifts from microsatellite (MS) insertion/deletions that are more than 8 amino acids long. Frameshifts of 8-60 amino acids long are very likely to include MHCI, MHCII and B-cell epitopes. Further, because of their increased immunogenicity, FS variants are much more likely to create both T- and B-cell responses. Therefore, fewer peptides are required to be screened to determine vaccine components. Point mutation neo-epitopes are unlikely to produce both B and T-cell responses. Second, the realm of FS space is much more restricted than that of all possible point mutations. This is particularly true for indels in microsatellites in coding regions. There are two possible FS that can be predicted from each of the ˜7000 microsatellites in coding regions. These numbers become smaller as putative peptides are filtered for restrictions for minimal length (e.g., >7aa) and the probability of eliciting immune responses. This makes it feasible to have a pre-existing set of FS peptides made that can be used to screen the immune cells, such as T-cells, of a patient for reactivity.

Peptides are produced and displayed in a number of ways. For example, in some embodiments the peptide candidates are synthesized and spotted on arrays. In some embodiments, arrays have about 100 selected FS peptides. In some embodiments, arrays have about 200 selected FS peptides. In some embodiments, arrays have about 300 selected FS peptides. In some embodiments, arrays have about 400 selected FS peptides. In some embodiments, arrays have about 500 selected FS peptides. In some embodiments, arrays have about 600 selected FS peptides. In some embodiments, arrays have about 700 selected FS peptides. In some embodiments, arrays have about 800 selected FS peptides. In some embodiments, arrays have about 900 selected FS peptides. In some embodiments, arrays have about 1000 selected FS peptides. In some embodiments, arrays have about 10,000 selected FS peptides. In some embodiments, arrays have about 20,000 selected FS peptides. In some embodiments, in-situ synthesis could produce an array having 1,000,000 or more peptides per array, or at least 1000, 10,000 or 100,000 peptides per array.

A T-cell response, in some embodiments, is important for killing cancer cells. Since the FS peptides are generally 8 aa or longer, it is very likely that a FS peptide will have a region that would bind to the patient's MHC to initiate an immune response. MHC binding can be predicted from commonly available algorithms. Alternatively, the blood sample from the patient could be screened for T-cell activity to the peptide candidates using a T cell assay, such as a proliferation assay, a cytokine assay, a cytotoxicity assay, a degranulation assay, flow cytometry, or combination thereof.

Methods of producing cancer vaccines disclosed herein not only provide much more relevant information for making a vaccine, but have benefits over the existing methods such as simplicity and cost efficacy. The methods do not require a biopsy of the tumor with the inherent cost, discomfort and danger. The assay itself is simpler, less expensive and faster than deep sequencing the tumor DNA. The mutations identified in the DNA are most likely to be the result of point mutations which have a low probability of being produced by a patient, or at least immunogenic. Current sequencing protocols are very poor at identifying insertions/deletions in microsatellites or mis-splicing events. The best source of peptides (FS) would be missed by current sequencing protocols. Sequencing a biopsy will only identify mutations in that part of the tumor. Mutations in other parts will be missed. By methods disclosed herein, all immune reactive parts of the tumor will be identified. The identification of vaccine components by sequencing will require the application of proprietary algorithms and other assays to identify potentially produced and immunogenic peptides. In contrast, this information is directly readout from our assay. While the broadest application may be for cancer, the same procedure would apply for a vaccine against other chronic diseases. Vaccines based on the mass spect (MS) FS herein eliminate cells that are not tumors but are aberrant for other reasons. Such aberrant, dysfunctional cells play a role in diseases such as diabetes, Alzheimer's disease, aging, autoimmune disease, chronic infections, and other diseases.

Methods herein, in some embodiments, comprise methods of frameshift variant development for inclusion in cancer vaccine development. Frameshift variants, as referred to herein, are alterations in an mRNA caused by errors in transcription, causing an insertion or deletion (indel) of one or two nucleotides in the mRNA or by mis-splicing of RNA resulting in a change in the amino acids of the resulting protein that are encoded after the frameshift variant. Methods of frameshift variant development herein include but are not limited to mRNA sequencing and array based hybridization. In some embodiments, frameshift peptides are developed by bioinformatics analysis of already available sequence data. FS variant peptides due to indels in MS can be directly inferred from the genome sequence data.

Methods herein, in some embodiments, screen a first population for peptides binding to antibodies or eliciting a T cell reaction in a biological sample from a set of subjects, wherein the first population of peptides comprises peptides encoded by a frameshifted mRNA expressed by a cancer cell. In some embodiments, each of the first population of peptides binds to at least one MHC subtype or elicit antibodies that bind the FS peptides. In some embodiments, a portion of the first population of peptides binds to more than one MHC subtype. In some embodiments, each of the first population of peptides binds to at least one HLA subtype. In some embodiments, each of the first population of peptides comprises at least one T cell epitope. In some embodiments, at least one of the first population of peptides comprises at least one T cell epitope. In some embodiments, each of the first population of peptides comprises at least on B cell epitope. In some embodiments, at least one of the first population of peptides comprises at least on B cell epitope. In some embodiments, first population of peptides is bound to a substrate. In some embodiments, mRNA sequencing for development of frameshift variants herein includes a method where mRNA from a tumor or cancer tissue is sequenced. In some embodiments, mRNA is purified from a tumor or cancer tissue from a patient. In some embodiments, mRNA is isolated from total mRNA from the tumor or cancer tissue or from blood. In some embodiments, mRNA is isolated using oligo-dT purification of total RNA. In some embodiments, mRNA is targeted for sequencing using an oligo-dT to prime the RNA sample. In some embodiments, the mRNA is amplified before sequencing. In some embodiments, the mRNA is amplified by PCR before sequencing. In some embodiments the mRNA is amplified by RT-PCR before sequencing. In some embodiments, mRNA sequencing comprises targeted sequencing of an mRNA having a microsatellite in the transcript. In some embodiments, mRNA is sequenced using at least one technique selected from Sanger sequencing, pyro-sequencing, ion semiconductor sequencing, polony sequencing, sequencing by ligation, nanoball sequencing, and single molecule sequencing.

Variants identified from mRNA sequencing are classified by type of variant. Variants may arise from mutations in DNA or alterations in the RNA during transcription or splicing herein, which include but are not limited to point mutations, silent mutations, insertions, deletions, cis-splicing errors, and trans-splicing errors. Of these, only insertions, deletions, cis-splicing errors, and trans-splicing errors are expected to lead to a frameshift in a protein produced from the variant mRNA. Confirmed frameshift variants are those that when translated produce a protein with a different amino acid sequence at more than one residue at residues C-terminal to the alteration. Frameshifted polypeptide sequences resulting from frameshift variants are assembled for further analysis.

In some embodiments, frameshift mutations are predicted based on microsatellite location in the genome. As transcripts having a microsatellite are more prone to transcription errors, frameshift polypeptides can be predicted to be resulting from an insertion or a deletion of one or two basepairs. Alternatively, frameshift polypeptides can be predicted by bioinformatics prediction of cis and/or trans splicing errors. A selection of all possible frameshift peptides can be assembled for further analysis.

Frameshifted polypeptide sequences, determined by mRNA sequencing or prediction, are further analyzed to determine immunoreactivity. In some embodiments, immunoreactivity is measured by MHC or HLA binding. In some embodiments, immunoreactivity is measured by antibody binding. In some embodiments, immunoreactivity is measured by T cell activity. In some embodiments, immunoreactivity is measured by antibody binding and T cell activity.

Binding to MHC is required for T cell activity and can be determined by binding assays. Alternatively, in silico methods of MHC binding are used to predict binding of a peptide to a MHC subtype. Data of peptides binding to MHC subtype molecules are used to develop binding prediction algorithms. These algorithms calculate scoring matrices that quantify the contribution of each residue in a fixed-length peptide to binding to an MHC molecule. Algorithms predict binding of a peptide to class I MHC or class II MHC. Algorithms to predict class I MHC binding include but are not limited to Artificial neural network (ANN), Stabilized matrix method (SMM), SMM with a Peptide:MHC Binding Energy Covariance matrix (SMMPMBEC), Scoring Matrices derived from Combinatorial Peptide Libraries (Comblib_Sidney2008), Consensus, NetMHCpan, NetMHCcons and PickPocket. Algorithms to predict class II MHC binding, include but are not limited to Consensus method, Combinatorial library, NN-align (netMHCII-2.2), SMM-align (netMHCII-1.1), Sturniolo, and NetMHCIIpan. The entire population of frameshift polypeptides is then scanned using one or more of the above algorithms for peptides binding to an MHC subtype molecule with a predicted affinity of IC50<500 nM.

Candidate frameshift peptides for cancer vaccines, in some embodiments, are screened for antibody reactivity in a subject needing treatment using a cancer vaccine. Antibody reactivity is determined using an assay for antibody binding to a peptide. In some embodiments, peptides for antibody screening are bound to a substrate, such as a plate, a glass slide, a bead, or other substrate. Assays for antibody binding include but are not limited to ELISA, radio-immuno assay, western blot, surface plasmon resonance, immunostaining, immunoprecipitation, mass spectrometry, phage display, flow cytometry, cytometric bead array, immunohistochemistry, high density array, microarray, and combinations thereof.

In some embodiments, candidate frameshift peptides are screened for T cell activity in cells obtained from a subject needing treatment using a cancer vaccine. T cell activity is determined using a T cell assay measuring proliferation, cytokine secretion, cytotoxicity, or degranulation in response to a frameshift peptide bound to an antigen presenting cell. T cell assays include but are not limited to proliferation assay, 3H-thymidine assay, BrdU assay, CFSE assay, cytokine secretion assay, ELISA assay, ELISPOT assay, intracellular staining assay, quantitative rtPCR assay, cytometric bead array assay, MHC-tetramer binding assay, cytotoxicity assay, 51-chromium assay, degranulation assay, granulysin assay, granzyme A assay, granzyme B assay, and perforin assay.

Vaccine Compositions and Formulations

The cancer vaccines disclosed herein (which can be used in the disclosed methods) comprise one or more peptides identified as immunoreactive with a set of biological samples obtained from a set of test subjects that have been identified as having the cancer and/or tumor of interest. For example, a cancer vaccines, in some embodiments, comprise 1, 2, 3, 4, 5, 6, 7, 8, 9, 10, 11, 12, 13, 14, 15, 16, 17, 18, 19, 20, or more frameshift peptides having an amino acid sequence determined to have immunoreactivity with the set of biological samples.

In some embodiments, a vaccine includes nucleic acids, such as on plasmids encoding the FS variants. DNA or Gene Vaccines typically include a plasmid with a promoter and appropriate transcription and translation control elements. The plasmids may also include sequences that encode peptide or protein fusions to the FS peptide to enhance, for example, expression levels, intracellular targeting or proteasomal processing. For example, the lysosome-associated membrane protein (LAMP) sequence when fused to a FS peptide sequence will enhance MHCII responses. In additional aspects, a cancer vaccine includes one or more nucleic acids encoding peptides identified as immunoreactive with a set of biological samples obtained from a set of test subjects that have been identified as having the cancer and/or tumor of interest. For example, in some embodiments, cancer vaccines comprise one or more nucleotide sequences encoding 1, 2, 3, 4, 5, 6, 7, 8, 9, 10, 11, 12, 13, 14, 15, 16, 17, 18, 19, 20, or more FS peptides identified as immunoreactive with a set of biological samples obtained from a set of test subjects that have been identified as having the cancer and/or tumor of interest. These plasmids can be introduced by a needle, a gene gun, an aerosol injector, with patches, via microneedles, by abrasion among many forms. In some forms, the DNA vaccine is incorporated into liposomes or other forms of nanobodies. They may also be administered by inhalation or ingestion. The plasmid can be introduced into the blood, the thymus, the pancreas, the skin, the muscle, a tumor or other sites.

In some embodiments, the peptides are encoded in RNA that is directly introduced into the subject. For example, the RNA is chemically synthesized or more commonly in vitro transcribed. The RNA will encode one or more FS peptides and will include signals to enhance stability and translation. The RNA may also include unnatural nucleotides to increase the half-life. These RNAs can be introduced by a needle, a gene gun, an aerosol injector, with patches, via microneedles, by abrasion among many forms. In some embodiments, the RNA is incorporated into liposomes or other forms of nanobodies. They may also be administered by inhalation or ingestion. The RNA can be introduced into the blood, the thymus, the pancreas, the skin, the muscle, a tumor or other sites

In some embodiments, the peptide coding sequences are introduced into a virus as a vector. The peptide encoding sequences are fused to other sequences that enhance transcription, translation or presentation to the immune system. These viral vectors include pox viruses, adenovirus, lentiviruses, retroviruses, alpha viruses and others using a needle, a gene gun, an aerosol injector, with patches, via microneedles, by abrasion among many forms. They may also be administered by inhalation or ingestion.

In some embodiments, the peptides are administered via a bacterial vector. The FS coding sequences are introduced into the bacteria, usually in the form of plasmid or lysogenic phage, and the bacteria administered to the patient. Listeria is commonly used in this way, but other bacteria may be used or developed. The bacteria can be administered by needle to the blood or intraperitoneal injection. Bacteria can also be administered orally.

In some embodiments, the peptides are delivered as peptides. Usually the peptides are 10aa long or longer, preferably 25aa or longer. 25-40aa long may be ideal. Usually the peptides are fused to a carrier such as albumin, keyhole limpet protein etc. In some embodiments, the peptides are incorporated into liposomes or other forms of nanobodies.

Vaccine formulations disclosed herein generally comprise a biologically acceptable formulation, gaseous, liquid or solid, or mixture thereof, which are suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

To increase an immune response and efficacy in treating cancer, the peptides are optionally coupled to another protein such as ovalbumin or keyhole limpet hemocyanin (KLH), thyroglobulin or a toxin such as tetanus or cholera toxin. In some embodiments, the peptides herein can be given with, such as separately or mixed with adjuvants and/or checkpoint inhibitors. Adjuvants include, for example: ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, Xtendlll, and Zymosan.

Checkpoint inhibitors are inhibitors of proteins known to potentiate or inhibit an immune response and accordingly a checkpoint inhibitor increases the immune response. In some instances, tumors are known to upregulate checkpoint inhibitors to evade the immune response in a patient and checkpoint inhibitors allow the immune system of the patient to recognize and eliminate the tumor. Checkpoint inhibitors herein include but are not limited to a PD-1 inhibitor, a PD-L1 inhibitor, a CTLA-4 inhibitor, Pembrolizumab, Nivolumab, or Atezolizumab. They may be administered systemically or locally as an adjuvant.

Cosolvents may be added to a frameshift peptide composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration include aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

Methods of Treatment

Provided herein is a method of eliciting an immune response in a subject in need thereof. In embodiments, the method includes selecting a subject with cancer or at risk for developing cancer. In embodiments, the method includes administering a disclosed vaccine composition to the subject, thereby eliciting an immune response in the subject in need thereof.

Disclosed is a method of reducing risk of developing cancer in a subject. In embodiments, the method includes selecting a subject with cancer or at risk for developing cancer. In embodiments, the method includes administering a disclosed vaccine composition to the subject, thereby reducing risk of developing cancer in the subject. Disclosed is a method of treating cancer and/or a tumor in a subject. In embodiments, the method includes selecting a treatment subject with cancer and/or a tumor. In embodiments, the method includes administering disclosed vaccine composition to the subject, thereby treating the cancer and/or tumor in the subject.

Methods herein comprise administration of one or more immunoreactive polypeptides reactive to immune cells and antibodies, for example from a subject with cancer. In some embodiments, immunoreactivity comprises antibody reactivity. In some embodiments, immunoreactivity comprises a T cell response. A T cell response herein includes but is not limited to one or more of proliferation assay, 3H-thymidine assay, BrdU assay, CFSE assay, cytokine secretion assay, ELISA assay, ELISPOT assay, intracellular staining assay, quantitative rtPCR assay, cytometric bead array assay, cytotoxicity assay, 51-chromium assay, degranulation assay, granulysin assay, granzyme A assay, granzyme B assay, and perforin assay.

In some embodiments, the methods are used to treat a cancer and/or tumor type selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of a disclosed composition for treating, inhibiting, or preventing a cancer, the efficacy or prophylaxis can be assessed in various ways well known to the skilled practitioner. For instance, one of ordinary skill in the art will understand that a composition, such as a vaccine, disclosed herein is efficacious in treating, inhibiting, or preventing a cancer in a subject by observing that the composition reduces tumor growth or prevents a further increase in tumor size.

Kits

The present disclosure also provides kits for treating a specific condition, such as a specific type of cancer. Such kits may include one or more containers comprising a formulation as described herein, which comprises a disclosed vaccine composition.

In some embodiments, the kit may comprise instructions for use in accordance with any of the methods described herein. The included instructions may comprise a description of administration of the formulation according to any of the methods described herein. The kit may further comprise a description of selecting an individual suitable for treatment based on identifying whether that individual has or is at risk of acquiring a particular condition, including cancer.

The instructions relating to the use generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for a particular condition or disease, including treating cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Kits may optionally provide additional components such as interpretive information. Normally, the kit comprises a container and a label or package insert(s) on or associated with the container. In some embodiments, the invention provides articles of manufacture comprising contents of the kits described above.

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLES

### Example 1

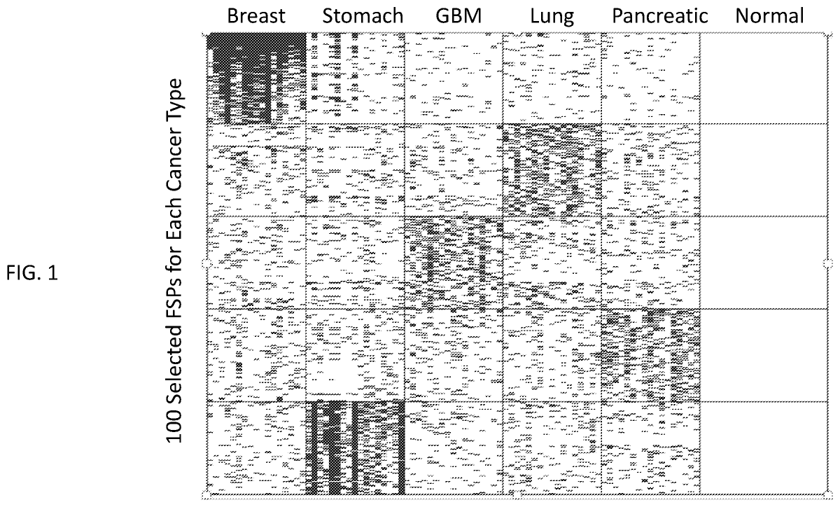

This example illustrates the ability to identify specific peptides associated with different cancers which can be used for developing cancer specific vaccines. 220K frameshift peptides (FSPs) that could be generated by exon mis-splicing and those from indels in micro-satellites were synthesized as 400K, 15mer peptides on arrays. The arrays were reacted with 10 μl of sera from patients with the cancer specified (15-17 patients/cancer type) and age-matched people without cancer. A peptide was chosen as positive if it had 6× the average florescence in the normal and was present in at least 20% of the patients with that cancer. The top 100 peptides for each cancer were selected and are shown in FIG. 1. 30 peptides for each cancer would be predicted to give 50% reactivity in at least 90% of all patients.

### Example 2

This example provides exemplary probe and frameshift antigen sequences that were used in vaccine development for glioblastoma multiforme (GBM; Tables 1a and 1b), Breast Cancer (BC; Tables 2a and 2b) or Lung Cancer (LC; Tables 3a and 3b). These sequences can be utilized to generate GBM, BC, or LC vaccines in accordance with the methods disclosed herein.

TABLE 1a
Glioblastoma Multiforme

| | SEQ ID | Positive rate in GBM and other cancers | | | | | | |
|---|---|---|---|---|---|---|---|---|
| PROBE_SEQENCE | NO | PROBE_ID | GBM | BC | Lung | PAN | GAS | NORMAL |
| TRTHQQSIQRPLS | SEQ ID | HCIM066811 | 52.9% | 11.8% | 17.6% | 5.9% | 47.1% | 0.0% |
| | NO: 1 | | | | | | | |
| SLTACLPMQGNTVLG | SEQ ID | HCIM060080 | 52.9% | 5.9% | 5.9% | 11.8% | 5.9% | 0.0% |
| | NO: 2 | | | | | | | |
| RSLQLPRGGALASAS | SEQ ID | HCIM368833 | 47.1% | 0.0% | 11.8% | 11.8% | 5.9% | 0.0% |
| | NO: 3 | | | | | | | |
| PPLSCGGRREGKRCF | SEQ ID | HCIM022910 | 47.1% | 11.8% | 0.0% | 0.0% | 0.0% | 0.0% |
| | NO: 4 | | | | | | | |
| ITLPHKCTTTNPATT | SEQ ID | HCIM222028 | 41.2% | 23.5% | 47.1% | 11.8% | 41.2% | 0.0% |
| | NO: 5 | | | | | | | |
| SKGFITSSQSPLQAF | SEQ ID | HCIM372257 | 41.2% | 11.8% | 35.3% | 17.6% | 17.6% | 0.0% |
| | NO: 6 | | | | | | | |
| SVSGARGWAQGGPRP | SEQ ID | HCIM002520 | 41.2% | 5.9% | 17.6% | 17.6% | 17.6% | 0.0% |
| | NO: 7 | | | | | | | |
| LASRSSSALGNSAWP | SEQ ID | HCIM181147 | 41.2% | 5.9% | 17.6% | 35.3% | 11.8% | 0.0% |
| | NO: 8 | | | | | | | |
| LTPRTSAATKALPRQ | SEQ ID | HCIM005423 | 41.2% | 11.8% | 11.8% | 17.6% | 11.8% | 0.0% |
| | NO: 9 | | | | | | | |
| HWDVRAGAPGEGEDS | SEQ ID | HCIM263381 | 41.2% | 5.9% | 11.8% | 0.0% | 11.8% | 0.0% |
| | NO: 10 | | | | | | | |
| PEPGTTSKWRLRTSQ | SEQ ID | HCIM270341 | 41.2% | 5.9% | 11.8% | 11.8% | 5.9% | 0.0% |
| | NO: 11 | | | | | | | |
| VPWTKMAGIVCASLD | SEQ ID | HCIM010851 | 41.2% | 17.6% | 5.9% | 11.8% | 35.3% | 0.0% |
| | NO: 12 | | | | | | | |
| CAKEAVHQFLPARPA | SEQ ID | HCIM379864 | 41.2% | 17.6% | 5.9% | 5.9% | 5.9% | 0.0% |
| | NO: 13 | | | | | | | |
| HKRQPNYVGTIISAT | SEQ ID | HCIM323455 | 41.2% | 11.8% | 5.9% | 5.9% | 5.9% | 0.0% |
| | NO: 14 | | | | | | | |
| PPRARPGPRARLPSG | SEQ ID | HCIM008224 | 41.2% | 11.8% | 0.0% | 5.9% | 0.0% | 0.0% |
| | NO: 15 | | | | | | | |
| GPTDGSFLPAVPVP | SEQ ID | HCIM016651 | 41.2% | 11.8% | 0.0% | 0.0% | 5.9% | 0.0% |
| | NO: 16 | | | | | | | |
| LGSTLCTSSVTMRTS | SEQ ID | HCIM090806 | 41.2% | 11.8% | 0.0% | 17.6% | 0.0% | 0.0% |
| | NO: 17 | | | | | | | |
| GLVVRDVHFRGPASM | SEQ ID | HCIM126137 | 35.3% | 5.9% | 35.3% | 23.5% | 35.3% | 0.0% |
| | NO: 18 | | | | | | | |
| GVVRVVGRRGPRTPV | SEQ ID | HCIM367936 | 35.3% | 5.9% | 29.4% | 17.6% | 17.6% | 0.0% |
| | NO: 19 | | | | | | | |
| NPTSVTAARPPSATR | SEQ ID | HCIM095751 | 35.3% | 29.4% | 23.5% | 5.9% | 5.9% | 0.0% |
| | NO: 20 | | | | | | | |

TABLE 1b
Glioblastoma Multiforme

| | Gene Name | SEQ ID | |
|---|---|---|---|
| FS antigen ID | or MS type | NO | FS antigen sequence |
| NM_001042683.2_Exon30_2nd | SHPRH | SEQ ID | TRTHQQSIQRPLS |
| | | NO: 21 | |
| NM_001037666.2_Exon9_3rd | CASTOR1 | SEQ ID | ARGRYRQRHRGPPAAAGRPGFLRPMGNRAASLL |
| | | NO: 22 | SPRPRLPKTSLSYFLKLWNEPPVLLGTLVPLSVCKLR |
| | | | AGTGFYADTCVHSQVEWAGASLTACLPMQGNIV |
| | | | LGA |
| NM_145867.1_Exon5_2nd | LTC4S | SEQ ID | WHRCTRARAPSGCWWRWLRSACSPTSSRPRCAP |
| | | NO: 23 | RSSDGSGRCCRGPETKAPGPTEPGKKSRSLQLPRG |
| | | | GALASAS |
| NM_000291.3_Exon4_3rd | PGK1 | SEQ ID | GCSVLEGLCRPRSGESLCQPSCWVCHPAGEPPLSC |
| | | NO: 24 | GGRREGKRCFWEQ |
| NM_001321079.1_Exon14_2nd | EZH1 | SEQ ID | ITLPHKCTTTNPATTQTAPVTAPAPAS |
| | | NO: 25 | |
| NM_152707.3_Exon2_3rd | SLC25A16 | SEQ ID | YCWMLCQNNSCSIGSSKGFITSSQSPLQAF |
| | | NO: 26 | |
| CCD513789.1 | CCCCCCC | SEQ ID | PPPQPAAVHGLCHPSSVSGARGWAQGGPRPPG |
| | | NO: 27 | APPSQRLRR |
| NM_001282867.1_Exon8_2nd | RHD | SEQ ID | LASRSSSALGNSAWPS |
| | | NO: 28 | |
| CCDS32830.1 | GGGGGGG | SEQ ID | GGRRMTRRTMTTTKMMMMRRTKRRRRKRRRM |
| | | NO: 29 | TMMTRRTLLTKKTCLTPRTSAATKALPRQTISQRR |
| | | | PIQTPPGTSLTPSRLAVLAIWG |
| NM_005044.4_Exon2_3rd | PRKX | SEQ ID | HWDVRAGAPGEGEDSQAFLRPQGDEHSRRHPPK |
| | | NO: 30 | AGATRTQ |
| NM_005755.2_Exon5_2nd | EBI3 | SEQ ID | WGPLKPRPSSSGLCGPEPGTTSKWRLRTSQTTGN |
| | | NO: 31 | |
| CCD547165.1 | GGGGGGG | SEQ ID | GGRVAVKKAGRAQPVIREPATPAVPSTGPARMAS |
| | | NO: 32 | VNAARAGMESTALSRVVLVCATAMEDVPWTKM |
| | | | AGIVCASLDGEEQAVT |
| NM_175840.2_Exon6_3rd | SMOX | SEQ ID | CAKEAVHQFLPARPAHREGGCHPPPGHWHHRQ |
| | | NO: 33 | DLSGIRGALLGP |
| NM_019601.3_Exon12_2nd | SUSD2 | SEQ ID | GPCTMRPPCSPTIPGSWSTTSCTNPSTTPPSSPSSP |
| | | NO: 34 | VRPPSTPAWHKRQPNYVGTIISATLMWQPLGA |
| CCD542523.1 | CCCCCCC | SEQ ID | PPRARPGPRARLPSGIPARLPRALQEAFGESEHPP |
| | | NO: 35 | AAALHGGPSAVL |
| NM_000063.5_Exon1_3rd | C2 | SEQ ID | GPTDGSFLPAVPVP |
| | | NO: 36 | |
| NM_001127487.1_Exon12_2nd | FLNC | SEQ ID | ASPSRGPHKPRSNVTTRGMAPAMCGTGPRSLGS |
| | | NO: 37 | TLCTSSVTMRTSETHPSLPTSCPPHLTASQ1 |
| NM_001168360.1_Exon1_3rd | PCDH11X | SEQ ID | GLVVRDVHFRGPASMRGVPLWRPGEKLHHPRRN |
| | | NO: 38 | ARKRPDRRLVERP |
| NM_145257.4_Exon2_3rd | CCSAP | SEQ ID | VPGERGEERVHEALPGAALGGVRAVLPRAAALPP |
| | | NO: 39 | RPPAAGAGARALALGRLGPGRLLGGLGVVRVVGR |
| | | | RGPRTPVRPALAPAARRAGDPGGGGTAGARGPG |
| | | | GAGRGGRGRGGRGRGGRGSA |
| NM_001130845.1_Exon7_2nd | BCL6 | SEQ ID | RTGPSSAMSVTAASLRRPHSRGTRCRPTVTNPTSV |
| | | NO: 40 | TAARPPSATRATSPATRPSIP |

TABLE 2a
Breast Cancer

| | SEQ ID | Positive rate in BC and other cancers | | | | | | |
|---|---|---|---|---|---|---|---|---|
| PROBE_SEQUENCE | NO | PROBE_ID | BC | Lung | GBM | PAN | GAS | NORMAL |
| GPRHAPSGCCPAAGQ | SEQ ID | HCIM018755 | 100.0% | 17.6% | 5.9% | 0.0% | 47.1% | 0.0% |
| | NO: 41 | | | | | | | |
| GPRRTAASLRAGPRT | SEQ ID | HCIM023044 | 100.0% | 17.6% | 11.8% | 17.6% | 35.3% | 0.0% |
| | NO: 42 | | | | | | | |
| GPRGLGAVGGARRCP | SEQ ID | HCIM047444 | 100.0% | 5.9% | 0.0% | 0.0% | 23.5% | 0.0% |
| | NO: 43 | | | | | | | |
| GPRGCAGAAALRPAG | SEQ ID | HCIM088836 | 100.0% | 17.6% | 0.0% | 11.8% | 17.6% | 0.0% |
| | NO: 44 | | | | | | | |
| GPRNSLRCACVPSPS | SEQ ID | HCIM095381 | 100.0% | 5.9% | 0.0% | 0.0% | 5.9% | 0.0% |
| | NO: 45 | | | | | | | |
| GPRAAASTQRNGPSW | SEQ ID | HCIM154402 | 100.0% | 29.4% | 17.6% | 11.8% | 35.3% | 0.0% |
| | NO: 46 | | | | | | | |
| GPRRVACLPAPRLTP | SEQ ID | HCIM163332 | 100.0% | 11.8% | 0.0% | 0.0% | 29.4% | 0.0% |
| | NO: 47 | | | | | | | |
| GPRGGGNWEGRVRRS | SEQ ID | HCIM178629 | 100.0% | 11.8% | 0.0% | 5.9% | 5.9% | 0.0% |
| | NO: 48 | | | | | | | |
| GPRGLLHPVPLPPNP | SEQ ID | HCIM204081 | 100.0% | 5.9% | 5.9% | 0.0% | 5.9% | 0.0% |
| | NO: 49 | | | | | | | |
| GPRLPPPPPVRPQLR | SEQ ID | HCIM221380 | 100.0% | 0.0% | 5.9% | 5.9% | 23.5% | 0.0% |
| | NO: 50 | | | | | | | |
| GPRTARRPVAASCAR | SEQ ID | HCIM240273 | 100.0% | 11.8% | 0.0% | 0.0% | 11.8% | 0.0% |
| | NO: 51 | | | | | | | |
| GPRGQDWKSSVFPRL | SEQ ID | HCIM327406 | 100.0% | 11.8% | 0.0% | 11.8% | 0.0% | 0.0% |
| | NO: 52 | | | | | | | |
| GPRRTQFQRASSLLS | SEQ ID | HCIM332931 | 100.0% | 0.0% | 17.6% | 11.8% | 5.9% | 0.0% |
| | NO: 53 | | | | | | | |
| GPRCLATGTPRSAPT | SEQ ID | HCIM338817 | 100.0% | 0.0% | 11.8% | 0.0% | 5.9% | 0.0% |
| | NO: 54 | | | | | | | |
| GPRASCSCCSHSLPN | SEQ ID | HCIM070218 | 94.1% | 5.9% | 0.0% | 5.9% | 5.9% | 0.0% |
| | NO: 55 | | | | | | | |
| GPRAQGGRNLPPPIL | SEQ ID | HCIM178193 | 94.1% | 17.6% | 0.0% | 5.9% | 17.6% | 0.0% |
| | NO: 56 | | | | | | | |
| GPRPNADPPQQLITI | SEQ ID | HCIM183725 | 94.1% | 23.5% | 0.0% | 0.0% | 35.3% | 0.0% |
| | NO: 57 | | | | | | | |
| GPRAGTRRSACSQT | SEQ ID | HCIM265215 | 94.1% | 5.9% | 11.8% | 11.8% | 35.3% | 0.0% |
| | NO: 58 | | | | | | | |
| GGRSALGTFAAATPP | SEQ ID | HCIM337437 | 94.1% | 5.9% | 17.6% | 0.0% | 29.4% | 0.0% |
| | NO: 59 | | | | | | | |
| GPRAVTCSSTICPRS | SEQ ID | HCIM054549 | 88.2% | 5.9% | 0.0% | 5.9% | 5.9% | 0.0% |
| | NO: 60 | | | | | | | |

TABLE 2b
Breast Cancer

| | Gene Name | SEQ ID | |
|---|---|---|---|
| FS antigen ID | or MS type | NO | FS antigen sequence |
| NM_000118.3_Exon1_3rd | ENG | SEQ ID | GPRHAPSGCCPAAGQLQPQPH |
| | | NO: 61 | |
| NM_000296.3_Exon10_2nd | PKD1 | SEQ ID | GPRRTAASLRAGPRTTGPSWPPRACQGDAGALE |
| | | NO: 62 | PTSACRWTPPATPRPAPMAARQGQGYPGPPMR |
| | | | YGESSSSPFPRGPPRST |
| NM_001010881.1_Exon16_3rd | C1orf167 | SEQ ID | GPRGLGAVGGARRCPASHPHPAPAG |
| | | NO: 63 | |
| NM_001122898.2_Exon1_3rd | CD99 | SEQ ID | GPRGCAGAAALRPAGCSGRRP |
| | | NO: 64 | |
| NM_001130677.1_Exon1_2nd | EPOP | SEQ ID | WRPCALRPAWQCRRPREGRPAPPRPGSRVGGPR |
| | | NO: 65 | NSLRCACVPSPSAPLPSPGRPLWARGLGSWRRGP |
| | | | QCCGALRPPCALAAGPRMA |
| NM_001244.3_Exon1_3rd | TNFSF8 | SEQ ID | GPRAAASTQRNGPSWRHSHACAGGLRGQPPGD |
| | | NO: 66 | HEPQLFLFDHSHSGSVPCLHGGHYYGVGRSED |
| NM_001258290.1_Exon5_3rd | PR5536 | SEQ ID | EWDAGARGRVVGTAGRALPGRAPGRRAHPRSG |
| | | NO: 67 | RHRGAGQLQPSGAGRRPGPAAPGLTRQPGPRRV |
| | | | ACLPAPRLTPLRARHRLLGHRLGRRPGG |
| NM_001282352.1_Exon13_3rd | ADAMTS10 | SEQ ID | VQGCEQGRLLPPGAQISVLQPSLLPPDVLQNLPW |
| | | NO: 68 | PLGGARHPEPQLAGSPPPALQRAGQRGPRGGGN |
| | | | WEGRVRRSRKLFIGNPCRALAGGMERGWLSPQS |
| | | | PSSASAPSSSHSETPPPGWVWGGDSCSPQHPLNP |
| | | | PLGSPQHSPPPLIGICWKSRGGEVVCRCPAPQHC |
| | | | PTPPLRAGGESVSAMGGGGVAPPP |
| CCD512716.1 | CCCCCCC | SEQ ID | PPPGPLIAGSQNGGVSQDVEPQGAPRLGPAVPRA |
| | | NO: 69 | LHSLLQGAAAPPPNTGIPLESGREGGFGGVLGPRG |
| | | | LLHPVPLPPNPGPAAGLI |
| NM_001320839.1_Exon4_3rd | DMAC2 | SEQ ID | PPEGAPVLVAAALLPRGRLVSQPPLPTGRLVAGAL |
| | | NO: 70 | AGRLPPHLRTGPRLPPPPPVRPQLRLGHMPRHLS |
| | | | HLTQMQERKWGGAMLGSSHIPLHPSQT |
| NM_002523.2_Exon1_2nd | NPTX2 | SEQ ID | CWRCWPPAWRSPWPLGPRTARRPVAASCARHC |
| | | NO: 71 | PQRRCTPAARCPRCPCRAARRVPRRS |
| NM_020721.1_Exon1_3rd | KIAA1210 | SEQ ID | EGRLDASRLLCLSRFSPSWPAPLPGSPGPQGPRG |
| | | NO: 72 | QDWKSSVFPRLLQLSLVDLQRQEGGKHQGGAWP |
| | | | SCSD |
| NM_021570.3_Exon4_2nd | BARX1 | SEQ ID | CCRAAAWSLPPSPRGGPRRTQFQRASSLLSRSAPR |
| | | NO: 73 | MQRNRRRCRASPATGAARTEGGIRCGAWDARA |
| | | | TRSPLTRRKPASRPFRVQEVYFLSLFIMILNADNW |
| | | | GQTRKDTDPKARPRSQRASGL |
| NM_023079.4_Exon1_2nd | UBE2Z | SEQ ID | WRRVRLRRRQRRAPGRRAPGRAALLVLLALAAAA |
| | | NO: 74 | AGSGRLSCRMCGRRRRQRAGPGARGAAWLRCP |
| | | | GSRPQPLPTGPRCLATGTPRSAPTGTASAPRRSVY |
| | | | SGSS |
| NM_001288820.1_Exon3_3rd | CPEB1 | SEQ ID | GGRVSDGPRASCSCCSHSLPNQCFKEMARSFCVA |
| | | NO: 75 | ILGPPRSSQRPLQHRERGQAAPTSC |
| NM_001282175.1_Exon11_3rd | CLPTM1 | SEQ ID | GPRAQGGRNLPPPILQGQVHVYRVLDQSV |
| | | NO: 76 | |
| NM_001286105.1_Exon3_2nd | CLN3 | SEQ ID | GPRPNADPPQQLITI |
| | | NO: 77 | |
| NM_005199.4_Exon2_2nd | CHRNG | SEQ ID | GPRAGTRRSACSQT |
| | | NO: 78 | |
| CCD514077.1 | GGGGGGG | SEQ ID | GGRSALGTFAAATPPAAAPSPTAPGTATGRSCTAL |
| | | NO: 79 | PSRSESTASWFDSLHEELKRIRSQTHTA |
| NM_001025087.1_Exon11_2nd | CELF4 | SEQ ID | GPRAVTCSSTICPRSLGTLS |
| | | NO: 80 | |

TABLE 3a
Lung Cancer

| | | Positive rate in Lung cancer | | | | | | |
|---|---|---|---|---|---|---|---|---|
| PROBE_ | SEQ | and other cancers | | | | | | |
| SE- | ID | PROBE_ | | | | | | NOR- |
| QUENCE | NO | ID | Lung | BC | GBM | PAN | GAS | MAL |
| RRTRRM | SEQ | HCIM | 52.9% | 23.5% | 11.8% | 11.8% | 17.6% | 0.0% |
| STRSTT | ID | 134644 | | | | | | |
| SPR | NO: | | | | | | | |
| | 81 | | | | | | | |
| CGRPPR | SEQ | HCIM | 52.9% | 23.5% | 17.6% | 11.8% | 17.6% | 0.0% |
| SSVRPS | ID | 178619 | | | | | | |
| ATA | NO: | | | | | | | |
| | 82 | | | | | | | |
| TYFPVK | SEQ | HCIM | 52.9% | 5.9% | 17.6% | 11.8% | 0.0% | 0.0% |
| GEGPRT | ID | 313408 | | | | | | |
| PLT | NO: | | | | | | | |
| | 83 | | | | | | | |
| IQSQPA | SEQ | HCIM | 47.1% | 41.2% | 23.5% | 23.5% | 41.2% | 0.0% |
| YLTMWN | ID | 311005 | | | | | | |
| IMM | NO: | | | | | | | |
| | 84 | | | | | | | |
| ITLPHK | SEQ | HCIM | 47.1% | 23.5% | 41.2% | 11.8% | 41.2% | 0.0% |
| CTTTNP | ID | 222028 | | | | | | |
| ATT | NO: | | | | | | | |
| | 85 | | | | | | | |
| QPGLLV | SEQ | HCIM | 47.1% | 23.5% | 5.9% | 5.9% | 41.2% | 0.0% |
| PGMYLS | ID | 304066 | | | | | | |
| PAQ | NO: | | | | | | | |
| | 86 | | | | | | | |
| GRDLLP | SEQ | HCIM | 47.1% | 17.6% | 5.9% | 11.8% | 35.3% | 0.0% |
| DQQAAD | ID | 022180 | | | | | | |
| PQP | NO: | | | | | | | |
| | 87 | | | | | | | |
| WLHPAS | SEQ | HCIM | 47.1% | 5.9% | 0.0% | 5.9% | 5.9% | 0.0% |
| RAVTAP | ID | 229577 | | | | | | |
| KSL | NO: | | | | | | | |
| | 88 | | | | | | | |
| STRTTR | SEQ | HCIM | 47.1% | 0.0% | 5.9% | 5.9% | 41.2% | 0.0% |
| ATGSNW | ID | 017950 | | | | | | |
| TPG | NO: | | | | | | | |
| | 89 | | | | | | | |
| QPALHG | SEQ | HCIM | 41.2% | 23.5% | 5.9% | 11.8% | 5.9% | 0.0% |
| GHGPTR | ID | 341807 | | | | | | |
| LPQ | NO: | | | | | | | |
| | 90 | | | | | | | |
| QTGKTQ | SEQ | HCIM | 41.2% | 17.6% | 0.0% | 29.4% | 5.9% | 0.0% |
| SRTGQS | ID | 300836 | | | | | | |
| WHV | NO: | | | | | | | |
| | 91 | | | | | | | |
| LLLLLL | SEQ | HCIM | 41.2% | 11.8% | 5.9% | 5.9% | 0.0% | 0.0% |
| SLDPNL | ID | 081140 | | | | | | |
| NPK | NO: | | | | | | | |
| | 92 | | | | | | | |
| TRRTDS | SEQ | HCIM | 41.2% | 11.8% | 0.0% | 0.0% | 5.9% | 0.0% |
| KSSRTG | ID | 164338 | | | | | | |
| SRT | NO: | | | | | | | |
| | 93 | | | | | | | |
| GSQHCF | SEQ | HCIM | 41.2% | 11.8% | 17.6% | 5.9% | 0.0% | 0.0% |
| CQSTVA | ID | 285274 | | | | | | |
| DLG | NO: | | | | | | | |
| | 94 | | | | | | | |
| SSTGNA | SEQ | HCIM | 41.2% | 11.8% | 0.0% | 35.3% | 17.6% | 0.0% |
| SLRCGR | ID | 306136 | | | | | | |
| PTS | NO: | | | | | | | |
| | 95 | | | | | | | |
| LDKKTR | SEQ | HCIM | 41.2% | 5.9% | 17.6% | 11.8% | 0.0% | 0.0% |
| LRVTSS | ID | 150019 | | | | | | |
| LSG | NO: | | | | | | | |
| | 96 | | | | | | | |
| EPSSWT | SEQ | HCIM | 41.2% | 5.9% | 5.9% | 17.6% | 0.0% | 0.0% |
| VSTWTL | ID | 203168 | | | | | | |
| KLE | NO: | | | | | | | |
| | 97 | | | | | | | |
| FYLATL | SEQ | HCIM | 41.2% | 5.9% | 5.9% | 5.9% | 0.0% | 0.0% |
| VEILEK | ID | 355908 | | | | | | |
| S | NO: | | | | | | | |
| | 98 | | | | | | | |
| TEKLYS | SEQ | HCIM | 41.2% | 5.9% | 5.9% | 5.9% | 11.8% | 0.0% |
| RTEREW | ID | 390272 | | | | | | |
| PGI | NO: | | | | | | | |
| | 99 | | | | | | | |
| VKALWL | SEQ | HCIM | 41.2% | 5.9% | 5.9% | 5.9% | 0.0% | 0.0% |
| KLNSGG | ID | 391647 | | | | | | |
| KEM | NO: | | | | | | | |
| | 100 | | | | | | | |

TABLE 3b
Lung Cancer

| | Gene Name | SEQ ID | |
|---|---|---|---|
| FS antigen ID | or MS type | NO | FS antigen sequence |
| NM_001185077.2_Exon2_2nd | ARHGDIA | SEQ ID | WLSRSPQPSSWPRLQRRTRRMSTRSTTSPRPR |
| | | NO: 101 | RASRRSRSWTRTTRACESTRRPCWAAWPFPQ |
| NM_001282352.1_Exon12_2nd | ADAMTS10 | SEQ ID | ALHSAASGSGSARCAAPATRARRRTSARRPCGR |
| | | NO: 102 | PPRSSVRPSATAQPPGTALK |
| NM_017559.2_Exon4_2nd | FNDC8 | SEQ ID | LQPWPLTSAAFLRTTPSRSPCGARNPGKRSCPS |
| | | NO: 103 | GRRRCGGWRIWNTYFPVKGEGPRTPLTYFQLQ |
| | | | PQALRNQSHETYHTTSTLRARGLAEWYGHPTP |
| | | | GLGPRLHRGPIHLEASQARPCQAQPLPTAARLP |
| | | | PPPNLGWV |
| NM_016441.2_Exon6_2nd | CRIM1 | SEQ ID | IQSQPAYLTMWNIMMETCFEWTTVGSVDAKG |
| | | NO: 104 | ALPSASPPSVVR |
| NM_001321079.1_Exon14_2nd | EZH1 | SEQ ID | ITLPHKCTTTNPATTQTAPVTAPAPAS |
| | | NO: 105 | |
| NM_015354.2_Exon39_3rd | NUP188 | SEQ ID | QPGLLVPGMYLSPAQSKDAAALLT |
| | | NO: 106 | |
| NM_000260.3_Exon28_3rd | MYO7A | SEQ ID | GRDLLPDQQAADPQPLQEQLCPGLDSRVSLRG |
| | | NO: 107 | LFRPLREVCQ |
| NM_001508.2_Exon1_2nd | GPR39 | SEQ ID | WLHPASRAVTAPKSLITVMSPSLRWPPGSKSPL |
| | | NO: 108 | FWCT |
| NM_000092.4_Exon5_3rd | COL4A4 | SEQ ID | STRTTRATGSNWTPGSPRTHWAFRRERNERGP |
| | | NO: 109 | RPSWSSRGQRR |
| NM_024690.2_Exon68_3rd | MUC16 | SEQ ID | RGGQRGAIHTELHHQQPALHGGHGPTRLPQV |
| | | NO: 110 | QHHRQRHAAP |
| NM_015156.3_Exon3_3rd | RCOR1 | SEQ ID | QTGKTQSRTGQSWHVGLVTQSKSVRSK |
| | | NO: 111 | |
| NM_001100164.1_Exon5_2nd | PHACTR2 | SEQ ID | RTLKTTLKHRQLLLYLLLLLLSLDPNLNPKNHLCL |
| | | NO: 112 | RKGPLLGPATKVMKCLPLKKIPRLLVSRPPSLHPS |
| | | | QQAETRPERL |
| NM_001260492.1_Exon11_3rd | RDX | SEQ ID | TRRTDSKSSRTGSRTKTSKRRSRTT |
| | | NO: 113 | |
| NM_012294.3_Exon21_3rd | RAPGEF5 | SEQ ID | LQSPEKPEFFLCHCDGSQHCFCQSTVADLG |
| | | NO: 114 | |
| NM_015557.2_Exon15_2nd | CHD5 | SEQ ID | ATPKGPTWLARPSPPSSTGNASLRCGRPTSTWS |
| | | NO: 115 | PTRGTRRAAR |
| NM_001212.3_Exon5_2nd | C1QBP | SEQ ID | LDKKTRLRVTSSLSGKLAFSPLASLNGRILIIHSTQI |
| | | NO: 116 | PWT |
| NM_001303242.1_Exon5_2nd | PRUNE1 | SEQ ID | EPSSWTVSTWTLKLERQPQRTANMWRN |
| | | NO: 117 | |
| NM_033225.5_Exon4_2nd | CSMD1 | SEQ ID | FYLATLVEILEKS |
| | | NO: 118 | |
| NM_206933.2_Exon62_3rd | USH2A | SEQ ID | NKPSSPPVRVRTIHNISHWCCGCKPCRRNFKPL |
| | | NO: 119 | DSDSNLRIFPKWTEKLYSRTEREWPGIATTVVRT |
| | | | YENQWCD |
| NM_207437.3_Exon8_2nd | DNAH10 | SEQ ID | VKALWLKLNSGGKEMQP |
| | | NO: 120 | |

### Example 3

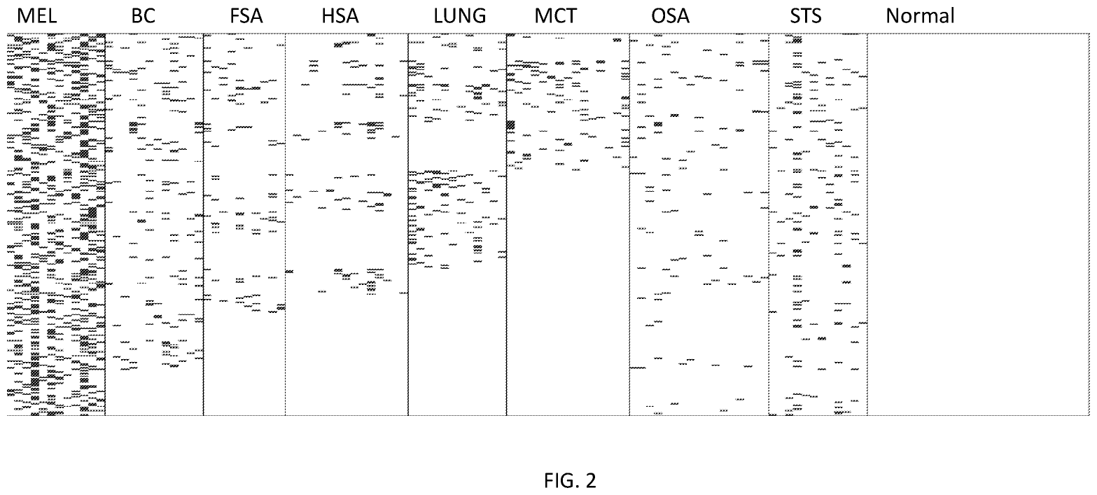

This example illustrates the ability to develop a frameshift vaccine for Myeloma (MEL) in dogs. 12-15 dogs with each of 8 different cancers and age-matched dogs without cancer, were assayed on the arrays as described in Example 1, except the frame-shift peptides were predicted for mis-splicing in dog tumors. 279 Myeloma specific frame-shift peptides (>25% positive rate, 6SD) are shown in FIG. 2. 30 peptides are predicted to be 50% reactive in >90% of dogs diagnosed with myeloma (MEL). The same process was applied to all 8 cancers in the same manner

### Example 4

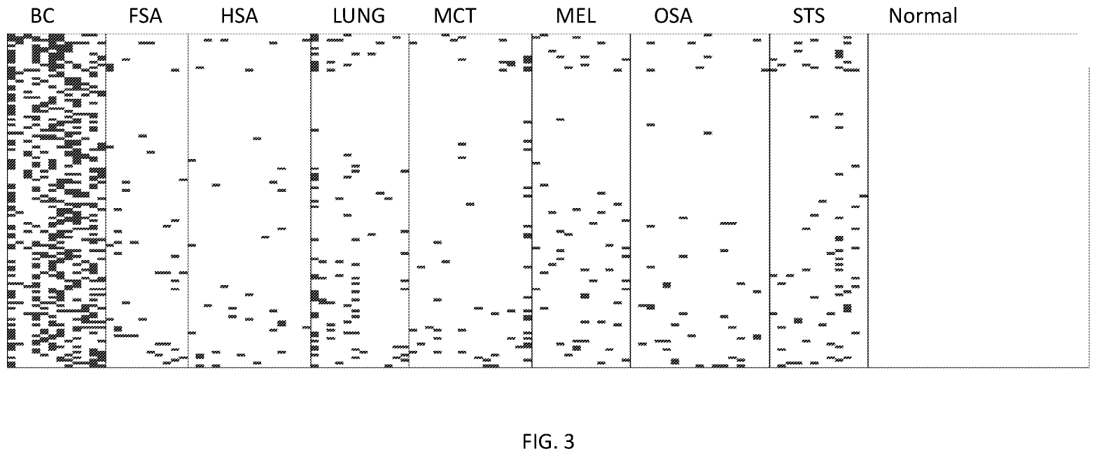

This example demonstrates the ability to develop a frameshift vaccine for Breast Cancer (BC) in dogs. 12-15 dogs with each of 8 different cancers and age-matched dogs without cancer, were assayed on the arrays as described in Example 1, except the frame-shift peptides were predicted for mis-splicing in dog tumors. 122 Breast Cancer specific frame-shift peptides (>30% positive rate, 6SD) are shown in FIG. 3. 30 peptides from this group are predicted to provide 50% reactivity in >90% of patients.

### Example 5

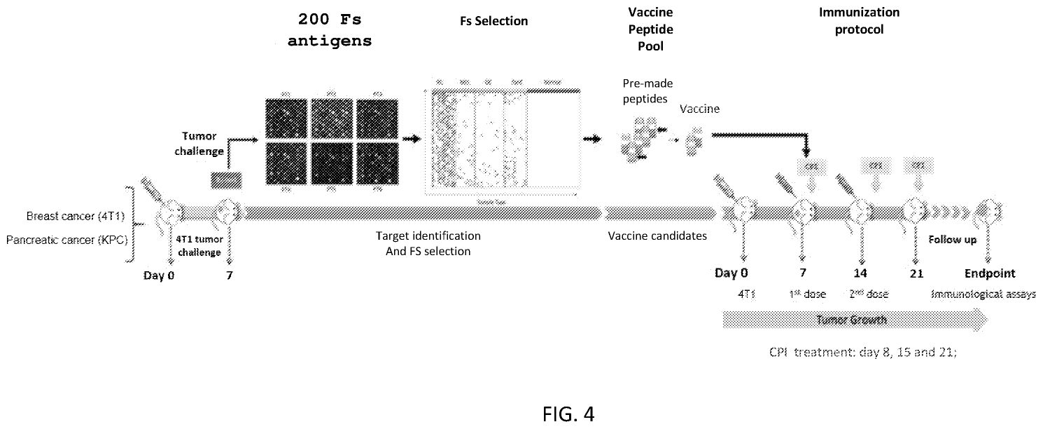

This example illustrates the process to test a cancer type specific frameshift vaccine in the mouse breast cancer model (4T1). As illustrated in FIG. 4, initially mice were challenged subcutaneously with 4T1 tumor cells and, seven days later, sera were collected. Pre-challenge and 7-days sera were assayed on peptide microarrays containing 200 frame-shift neoantigens. For the frameshift vaccines, the top 10 candidates with higher prevalence among all the mice challenged (n=24) were selected, a common Breast cancer frameshift vaccine was constructed (mBC FAST vaccine). Then, mice were vaccinated (n=10/group) twice subcutaneously on 7 and 14. For the immunization group with the combined immunotherapy, the antibody treatment, anti-PD-L1 (200 μg/dose) and CTLA-4 (100 μg/dose) were administrated on days 8, 15 and 21.

### Example 6

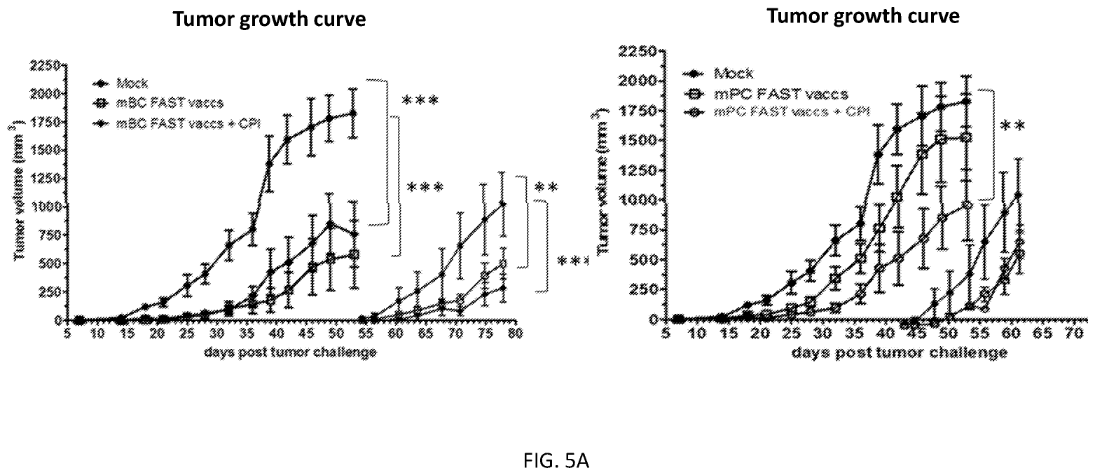

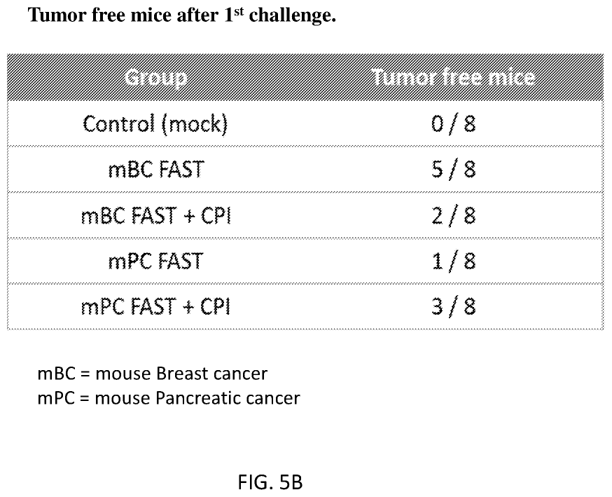

This example illustrates the Breast Cancer (BC) frameshift vaccine or Pancreatic Cancer (PC) frameshift vaccine confers protection by reducing tumor growth or preventing growth at all. As shown in FIGS. 5A and 5B, the vaccines produced by the methods disclosed herein are effective at reducing or preventing tumor growth. In particular, the BC FAST vaccine significantly reduced tumor growth either alone or in combination with check point inhibitor (CPI, see FIG. 5A). The BC FAST vaccine also resulted in 5 out of 8 mice to be tumor free after the 1^{st} challenge (see FIG. 5B). The PC FAST vaccine significantly reduced tumor growth in combination with CPI (FIG. 5A) and the combination resulted in 3 out of 8 mice to be tumor free after the 1^{st} challenge (see FIG. 5B).

### Example 7

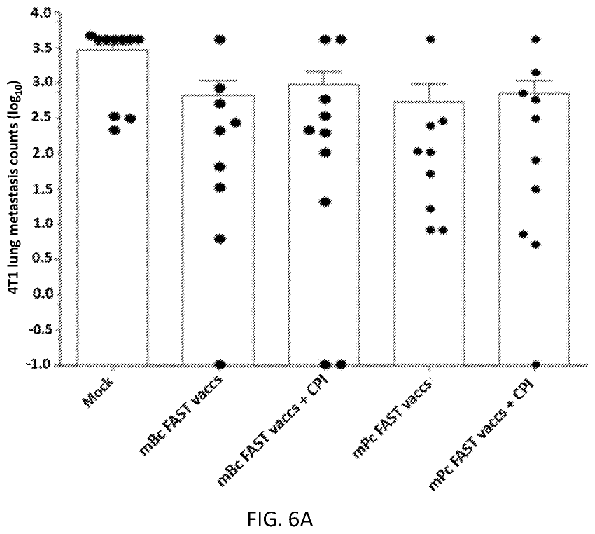

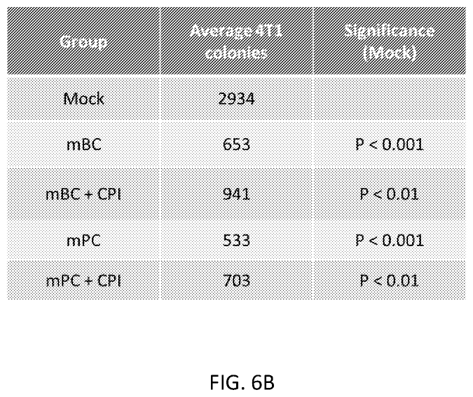

This example illustrates the disclosed FAST vaccine reduces metastasis, such as pulmonary metastasis. As illustrated in FIGS. 6A and 6B, treatment with either the BC FAST vaccine alone or with CPI significantly reduced metastasis. The average number of 4T1 colonies were significantly reduced following treatment with the BC FAST vaccine alone or in combination with CPI as compared to mock samples (FIG. 6B). Similar results were found following treatment with the BC FAST vaccine alone or in combination with CPI as compared to mock samples (FIG. 6B).

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method of producing a vaccine for a cancer and/or tumor and stage of interest, comprising:
identifying a first population of peptides that are immunoreactive with a set of biological samples obtained from a set of test subjects that have been identified as having the cancer and/or tumor and stage of interest; and
preparing a cancer vaccine composition for the cancer of interest, wherein the cancer vaccine composition comprises a second population of peptides comprising one or more peptides in the first population or a nucleic acid sequence encoding the one or more peptides, thereby producing the vaccine for the cancer and/or tumor of interest.

2. The method of claim 1, wherein the second population of peptides is a subpopulation of the first population.

3. The method of any one of claim 1 or claim 2, wherein identifying first population of peptides comprises determining immunoreactivity of the first population of peptides to the set of biological samples obtained from the set test subjects using antibody reactivity.

4. The method of claim 3, wherein antibody reactivity is detected using an antibody assay that comprises ELISA, radio-immunoassay, western blot, surface plasmon resonance, immunostaining, immunoprecipitation, mass spectrometry, phage display, ELISPOT, flow cytometry, cytometric bead array, immunohistochemistry, high density array, microarray, delayed-type hypersensitivity (DTH), and combinations thereof.

5. The method of any one of claims 1 to 4, wherein identifying comprises determining immunoreactivity of the first population of peptides to the set of biological samples obtained from the set test subjects using a T cell response.

6. The method of claim 5, wherein the T cell response is detected using a proliferation assay, 3H-thymidine assay, BrdU assay, CFSE assay, cytokine secretion assay, ELISA assay, ELISPOT assay, intracellular staining assay, quantitative rtPCR assay, cytometric bead array assay, cytotoxicity assay, 51-chromium assay, degranulation assay, granulysin assay, granzyme A assay, granzyme B assay, and/or perforin assay.

7. The method of any one of claims 1 to 6, wherein the first population of peptides comprises peptides encoded by a frameshifted mRNA expressed by a cancer cell.

8. The method of claim 7, wherein the frameshifted mRNA is created in a splicing error or a transcription insertion or deletion error.

9. The method of any one of claims 1 to 8, wherein each of the first population of peptides binds to at least one MHC subtype.

10. The method of any one of claims 1 to 9, wherein each of the first population of peptides comprises at least one T cell epitope.

11. The method of any one of claims 1 to 10, wherein each of the first population of peptides comprises at least on B cell epitope.

12. The method of any one of claims 1 to 11 wherein the first population of peptides is bound to a substrate.

13. The method of any one of claims 1 to 12, wherein the first population of peptides is part of an array or a phage display library.

14. The method of any one of claims 1 to 13, wherein the vaccine composition further comprises a pharmaceutically acceptable adjuvant or excipient.

15. The method of any one of claims 1 to 14, wherein the vaccine composition further comprises an immune checkpoint inhibitor or immune modulator.

16. The method of any one of claims 1 to 15, wherein the set of biological samples obtained from the set test subjects comprises one or more of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor.

17. The method of any one of claims 1 to 16, further comprising administering an effective amount of the vaccine composition to a treatment subject in need thereof to treat or prevent the cancer and/or tumor.

18. The method of claim 17, wherein treating the cancer and/or tumor comprises reducing tumor size, inhibiting tumor growth, reducing tumor burden, decreasing metastasis, increasing survival, or increasing cancer-free survival.

19. The method of any one of claims 17 and 18, wherein administering the vaccine composition elicits an immune response in the subject against the cancer.

20. The method of any one of claims 1 to 19, wherein the cancer is selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

21. A vaccine composition produced by the method of any of claims 1-20.

22. A method of eliciting an immune response in a treatment subject in need thereof, comprising:
selecting a treatment subject with cancer or at risk for developing cancer; and
administering the vaccine composition of claim 21 to the subject, thereby eliciting an immune response in the subject in need thereof.

23. A method of reducing risk of developing cancer in a subject comprising:
selecting a treatment subject with cancer or at risk for developing cancer; and
administering the vaccine composition of claim 21 to the subject, thereby reducing risk of developing cancer in the subject.

24. A method of treating cancer and/or a tumor in a subject comprising:
selecting a treatment subject with cancer and/or a tumor; and
administering the vaccine composition of claim 21 to the subject, thereby treating the cancer and/or tumor in the subject.
